(19) United States
(12) Patent Application Publication
(10) Pub. No.: US 2010/0197740 A1
(43) Pub. Date: Aug. 5, 2010

(54) Method of Screening Compounds Having Anti-Amyloid Properties

(75) Inventors: Hoau-Yan Wang, Philadelphia, PA (US); Philippe Morain, Issy Les Moulineaux (FR); Caryn Thibierge, Bois Colombes (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, Suresnes Cedex (FR)

(21) Appl. No.: 12/310,270

(22) PCT Filed: Aug. 16, 2007
(86) PCT No.: PCT/FR2007/001372

§ 371 Date: Mar. 25, 2010

(30) Foreign Application Priority Data
Aug. 18, 2006 (FR) 06/07385

(51) Int. Cl. A61K 31/44; C07D 213/26; A61P 25/28; G01N 33/566

(52) U.S. Cl. 514/354; 546/314; 436/501

(57) ABSTRACT

The invention relates to a method of screening compounds having anti-amyloid properties. The method of screening compounds that are capable of dissociating or preventing high-affinity complexes between β-amyloid peptides and nicotinic acetylcholine receptors of human cortical tissues makes it possible to rapidly identify compounds intended for the curative and/or preventive treatment of neurodegenerative diseases, especially Alzheimer's disease.

The present invention concerns the medical field and is of interest especially to pharmacological research units. The invention relates in fact to a method of screening compounds having anti-amyloid properties.

To that end, the invention employs biochemical techniques for the ex vivo analysis of biological samples allowing the rapid identification of compounds intended for the curative and/or preventive treatment of neurodegenerative diseases, especially Alzheimer's disease. The invention accordingly relates to a method of screening compounds that are capable of dissociating high-affinity complexes between the β-amyloid peptide and the nicotinic acetylcholine receptor of human cortical tissues.

Alzheimer's disease is a progressive neurodegenerative disease which affects a large proportion of the elderly population. In clinical terms, the disease is characterised by a loss of memory and a decline in cognitive functions. In neuropathological terms, Alzheimer's disease manifests itself in the presence of two types of histopathological cerebral lesions: amyloid plaques and neurofibrillary degeneration (NFD). A third characteristic of Alzheimer's disease is the cortical atrophy which corresponds to pronounced neuronal loss.

The accumulation of β-amyloid (Aβ) peptides in the form of intraneuronal deposits and of amyloid plaques, or senile plaques, around the neurons is considered to lie at the origin of the aetiology of Alzheimer's disease. In conjunction with the associated cognitive disturbances and the neurofibrillary degeneration, the accumulation of amyloid deposits represents the early and unvarying feature of all forms of Alzheimer's disease, including the familial forms.

Neurofibrillary degeneration corresponds to an intraneuronal accumulation of fibrils formed of paired helical filaments or PHFs. PHFs are composed of an assemblage of tau microtubule-associated proteins. Biochemical characterisation of these proteins reveals the presence of a major triplet of abnormally phosphorylated and aggregated tau proteins (tau 60, 64 and 69). Normal tau protein is phosphorylated 2 to 3 times, as opposed to 5 to 9 times in Alzheimer's disease, and it plays a part in the polymerisation/depolymerisation of microtubules of the neuronal cytoskeleton and also in axonal transport.

Cortical atrophy manifests itself in Alzheimer's disease patients in a loss of 8 to 10% of the weight of the brain every ten years, whereas in healthy subjects that loss is only 2%. Cortical atrophy is accompanied by dilatation of the cerebral ventricles and of the cortical sulci, by a reduction in the volume of the hippocampus and also by neuronal loss which especially affects the cholinergic system.

Amyloid plaques result from globular deposits of amyloid substance. Amyloid substance is composed of filaments of a polypeptide of 39 to 43 amino acids known as Aβ (β-amyloid). The β-amyloid peptide has a beta-sheet structure giving it its insoluble character and its toxicity. The β-amyloid peptide is a normal catabolic product of a membrane glycoprotein of large size known as APP (amyloid precursor protein). The amyloid plaques are surrounded by neuritic processes and glial cells. The amyloid plaques infiltrate the nervous parenchyma and diffuse into the cortical grey matter of all regions of the brain. The occipital cortex seems to be more frequently affected by these amyloid deposits. The neurotoxicity of the β-amyloid peptide constitutes a major problem in Alzheimer's disease.

Recent studies show that the amyloid plaques located in the extracellular space are the result of cell lysis of neurons having a very substantial accumulation of amyloid deposits in the lysosomal compartment. This intraneuronal accumulation causes degeneration of the neuronal cell and then cell death and release of those deposits into the extracellular space, gradually forming the amyloid plaques (Nagele et al. 2002). The amyloid plaques are surrounded by neuritic processes and glial cells and contain fragments of nuclei (evidence that the plaques result from dead neurons). The α7 type nicotinic receptor plays a fundamental role in entry of the Aβ peptide into the neurons (D'Andrea and Nagele 2006).

Wang et al. have shown that the Aβ peptide binds specifically and with high affinity to the α7 nicotinic acetylcholine receptors (α7 nAChR) present on the extracellular surface of the neuron (Wang et al. 2000). Interaction of the Aβ peptide, especially the Aβ₄₂ peptide, with the α7 nAChR receptor seems to be an essential step prior to the intraneuronal accumulation of the Aβ₄₂-α7 nAChR complexes, said complexes on the surface of the neurons undergoing endocytosis which results in their accumulation in the lysosomal compartment (Nagele et al. 2002).

In addition, the intraneuronal accumulation of those Aβ-α7 compounds causes abnormal phosphorylation of tau (Wang et al. 2003) and synaptic dysfunctions including a failure of cholinergic neurotransmission (Roselli et al. 2005; Almeida et al. 2005; Shemer et al. 2006).

This data, taken as a whole, tends to show that chronic disruption of the α7 nAChR receptors by Aβ peptides, especially Aβ₄₂ peptides, in the elderly and those suffering from Alzheimer's disease is a central mechanism by which Aβ peptides bring about neuronal dysfunctions, the formation of amyloid plaques and the phosphorylation of tau proteins lying at the origin of fibrillary neurodegeneration.

As a consequence, compounds that are capable of inhibiting the Aβ₄₂-α7 nAChR interaction could prove to be especially effective agents for reducing the formation of amyloid plaques and neuronal dysfunctions.

It accordingly appears to be of value, in the light of their importance in neurodegenerative and age-related pathologies, to identify compounds capable of acting on the Aβ₄₂-α7 nAChR complex, which lies at the origin of the formation of amyloid plaques.

Identification of these compounds can be carried out by various methods which, depending on the situation, are found to be more or less suitable and efficient. They are sometimes inadequate on their own and are then useful only when combined and, in any event, they have a certain number of advantages and drawbacks which are summarised hereinbelow and which will be discussed on the basis of two animal model validity criteria: construct validity, which is based on the similarity in the causative conditions of the pathology and the underlying neurobiological mechanisms; and descriptive validity, which is based on the similarity in the behavioural states that are brought about.

A first method consists of an injection of β-amyloid proteins into the brains of mice, carried out using a cannula in an intra-cerebro-ventricular (i.c.v.) position. This method (Yamada et al. 2005; Mazzola et al. 2003) makes it possible to obtain mice having a memory deficit after 7 days' exogenous introduction of β-amyloid peptides. This murine model is rapidly obtained and can be used for testing new prospective substances for the treatment of neurodegenerative pathologies, especially Alzheimer's disease. This method is based on a model which does not exactly represent the pathophysiology of Alzheimer's disease. In fact, this method of identifying compounds acting on the Aβ₄₂-α7 nAChR complex has no apparent validity because the tau pathology of cerebral ageing does not develop in this murine model. In addition, the present murine model does not satisfy construct validity in view of the fact that, on the one hand, the β-amyloid peptides are of exogenous origin and not naturally produced by the animal and, on the other hand, the model is animal and not human. Finally, the injection of exogenous β-amyloid peptides into the brains of mice makes it necessary to work in vivo, which precludes this method from being used routinely for screening and identifying anti-Alzheimer compounds.

Several other methods of identifying compounds use, as models of Alzheimer's disease, transgenic mice which carry mutations that are present in the familial forms of Alzheimer's disease, on the APP and/or PS1 (presenilin-1) genes. The construct validity of these models is accordingly undeniable for the familial forms but highly debatable for the sporadic forms, which represent more than 97% of cases.

A first type of transgenic mouse has just one mutation in APP (Hsiao et al. 1996) or a double mutation in APP and PS1 (Holcomb et al. 1998). The descriptive validity of the above-described transgenic models is not complete because they do not reliably reproduce the physiopathological features associated with Alzheimer's disease, there being found, on the one hand, an absence of neurofibrillary degeneration and, on the other hand, little or no neuronal loss and also the tardy appearance of senile plaques in the cortex of the single or double transgenic mice. As a consequence, in view of the physiological differences vis-a-vis Alzheimer's disease and the time that it takes for the lesions associated with this pathology to appear, the use of single or double transgenic mouse models is not recommended.

The use of a transgenic model of mice harbouring three mutant genes (APP, PS1 and tau) (LaFerla et al. 2003) also has disadvantages. The construct validity of this model is debatable because, compared to the preceding models, an additional mutation, not present in humans suffering from Alzheimer's disease, is added in the tau gene. However, the descriptive validity of this model is good because it mimics well the physiological lesions of Alzheimer's disease, which consist of amyloid plaques, neurofibrillary degeneration and neuronal loss. However, this method requires a period of 6 months to 12 months before mice are obtained that have the lesions typical of Alzheimer's disease. As a consequence, this transgenic mouse model can be validly used in a method for confirming the anti-amyloid properties of a tested compound but, in view of the time taken for said model and the difficulty in carrying it out, it cannot reasonably be used for screening compounds in the first instance.

A third method (Wang et al. 2000) consists of testing the ability of compounds to prevent the formation of complexes between exogenously introduced Aβ peptides and α7 present in rat tissues (rat hippocampus and cortex synaptosomes). This in vitro method is quicker to perform than the above-described methods, but it does have the drawback of not being representative of the complexes that are present in humans, because rat brain extracts are used and the Aβ peptides are exogenously introduced. As a consequence, this model satisfies neither the conditions for construct validity nor those for descriptive validity that are required for using this model in a method of screening compounds capable of acting on the Aβ₄₂-α7 nAChR complex, which lies at the origin of the formation of amyloid plaques.

The present invention accordingly has the objective of proposing an alternative strategy to the methods of identifying compounds capable of acting on β-amyloid-α7 nAChR complexes, with a view to overcoming, at least in part, the known drawbacks of the methods of selecting said compounds. To that effect, the invention accordingly proposes a method of ex vivo screening which recreates the physiological conditions present in Alzheimer's disease patients. These optimal conditions are obtained by using human brains, especially frontal cortices obtained from Alzheimer's disease patients. By definition, this model meets the criteria of construct validity and descriptive validity because it involves directly using diseased human tissue.

The ex vivo conditions of the screening method according to the invention make it possible to avoid the constraints associated with the implementing and handling of animal models. Moreover, the use of human biological material makes it possible to avoid all the artefacts and errors associated with the physiological differences that exist between animal species and humans. The use of human biological material in the context of the screening method according to the invention is especially important in view of the fact that Alzheimer's disease and neurodegenerative pathologies in general do not exist naturally in species other than humans.

The invention accordingly relates to a method of screening compounds that are capable of dissociating or preventing complexes of β-amyloid peptides with nicotinic acetylcholine receptors derived from human brains.

The invention relates preferably to a method of screening compounds that are capable of dissociating or preventing complexes of β-amyloid peptides with α7 nicotinic acetylcholine receptors derived from human brains.

The screening method according to the invention accordingly makes it possible to identify compounds that have curative or preventive properties, depending on whether said compounds are capable of dissociating or of preventing, respectively, the Aβ₄₂-α7 nAChR complexes.

An “anti-amyloid” or “anti-beta-amyloid” property is understood to be the ability of a compound to dissociate—or oppose the formation of—intracellular or extracellular deposits of β-amyloid peptides, be it by means of dissociating—or by means of inhibiting the formation of—the complexes that are formed by Aβ peptides with nicotinic acetylcholine receptors.

In the context of the invention the alpha-7 nicotinic acetylcholine receptor (α7 nAChR) denotes a cellular receptor having a pentameric surface, which is expressed principally in the cortex and hippocampus and which has an important role in learning and memory.

In the context of the invention, the expressions “β-amyloid”, “Aβ” and “β-amyloid peptide” relate to the entirety of β-amyloid peptides, including the peptides Aβ₁₋₃₉ or Aβ₃₉, Aβ₁₋₄₀ or Aβ₄₀, Aβ₁₋₄₁ or Aβ₄₁, Aβ₁₋₄₂ or Aβ₄₂, Aβ₁₋₄₃ or Aβ₄₃, and fragments thereof (Glenner et al. 1984). The above β-amyloid peptide fragments have biological activity and can be used in the screening method according to the present invention. Said fragments are, for example, fragments Aβ₁₋₂₈ and Aβ₂₅₋₃₅.

The β-amyloid peptides used in the context of the invention are especially the peptides Aβ₃₉, Aβ₄₀, Aβ₄₁, Aβ₄₂ and/or Aβ₄₃. The peptide Aβ₄₂ has the greatest affinity for α7 nicotinic acetylcholine receptors and the most important role in the aetiology of Alzheimer's disease.

The present screening method is carried out using samples from human brain, preferably human cortex and hippocampus. These samples are taken post-mortem from Alzheimer's disease patients.

The invention preferably relates to a screening method characterised in that the dissociation of complexes of β-amyloid peptides and α7 nicotinic acetylcholine receptors is demonstrated by immunohistochemistry.

In the context of the invention, the term “immunohistochemistry” relates to the entirety of antigen-revealing techniques using antibodies for the detection or isolation of defined molecules.

The screening method preferably comprises the following steps of: incubation of complexes of β-amyloid peptides with nicotinic acetylcholine receptors in the presence or absence of a compound under test and then determination of the amount of non-dissociated complexes in the presence or absence of compound under test and evaluation of the difference in amount of non-dissociated complexes, that difference indicating that the compound under test mediates the dissociation of complexes of β-amyloid peptides with nicotinic acetylcholine receptors.

The screening method according to the invention preferably also comprises a step of isolation of the non-dissociated complexes of β-amyloid peptides with nicotinic acetylcholine receptors using anti-β-amyloid peptide antibodies.

The anti-β-amyloid peptide antibodies used in the screening method are preferably directed to the β-amyloid peptides Aβ₃₉, Aβ₄₀, Aβ₄₁, Aβ₄₂ and/or Aβ₄₃. These antibodies can be mouse or goat monoclonal antibodies.

Even more preferably, the present screening method is characterised by revealing, especially by a Western blot method, non-dissociated complexes using anti-nicotinic acetylcholine receptor antibodies, especially anti-α7 nicotinic acetylcholine receptor antibodies.

Advantageously, the screening method according to the invention has shown the compound S 24795, i.e. 1-(4-bromophenyl)-2-(1-methyl-2-pyridiniumyl)-1-ethanone chloride or iodide, to be a compound capable, on the one hand, of inhibiting the formation of β-amyloid-α7 nAChR complexes and, on the other hand, of dissociating said β-amyloid-α7 nAChR complexes that have accumulated in amyloid plaques around the neuron and been deposited inside the neuron. The compound S 24795 identified by the screening method of the present invention is accordingly a compound capable of dissociating the complexes of β-amyloid peptides with nicotinic acetylcholine receptors present in the brains of Alzheimer's disease patients and also of inhibiting the formation of said complexes.

The invention relates also to each compound identified using the screening method according to the invention.

The invention relates also to a pharmaceutical composition comprising, as active ingredient, the compound obtained using the screening method according to the invention, in combination with one or more pharmaceutically acceptable excipient(s).

An “active ingredient” is understood to be any substance responsible for the pharmacodynamic or therapeutic properties of the pharmaceutical composition. In the context of the invention, “excipients” are understood to be any substance with which the active ingredient of a medicament is incorporated in order to facilitate its preparation and administration and to modify its consistency, form and volume.

Among the non-toxic, pharmaceutically acceptable excipients there may be mentioned, by way of example and without implying any limitation, diluents, solvents, preservatives, wetting agents, emulsifiers, dispersants, binders, swelling agents, disintegrants, retardants, lubricants, absorbency agents, suspension agents, colourants and flavourings.

Furthermore, the pharmaceutical compositions intended for the prevention and/or treatment of neurodegenerative pathologies, especially Alzheimer's disease, are in a form suitable for oral, parenteral, nasal, per- or trans-cutaneous, rectal, perlingual, ocular or respiratory administration, especially tablets or dragées, sublingual tablets, sachets, paquets, capsules, glossettes, lozenges, suppositories, creams, ointments, dermal gels and injectable or drinkable ampoules.

The present invention further relates to use of compounds identified using the screening method according to the invention in obtaining pharmaceutical compositions intended for the prevention and/or treatment of neurodegenerative diseases.

The compounds identified by the screening method according to the invention are used in the treatment of “neurodegenerative pathologies” such as, for example, Alzheimer's disease, Pick's disease, Lewy body dementia, Steele-Richardson syndrome, Down syndrome, Shy-Drager syndrome, amyotrophic lateral sclerosis, neurodegenerative ataxia, Huntington's disease, Parkinson's disease, primary progressive aphasia, Machado-Joseph disease, Tourette syndrome, paralytic dysarthria, Kennedy's disease, familial spasmodic paralysis, Werdnig-Hoffmann disease, Kugelberg-Welander disease, Tay-Sachs disease, Sandhoff disease, Wohlfart-Kugelberg-Welander disease, spastic paraparesis, progressive multifocal leukoencephalitis and prion-related diseases including Creutzfeldt-Jakob and Gerstmann-Sträussler-Scheinker disease.

The term “preventive” in accordance with the invention corresponds to preventively directed treatment having the objective of reducing the risk of developing Alzheimer's disease by inhibiting the binding of β-amyloid peptides to α7 nicotinic acetylcholine receptors. This inhibition limits the formation of β-amyloid-α7 nAChR complexes, which lie at the origin of amyloid plaques, which are lesions present in Alzheimer's disease. In addition, the term “preventive” can be understood as secondary prevention, which is intended to reduce prevalence by reducing the progression and duration of the disease.

“Treatment” is understood as being curatively directed treatment prescribed for the purpose of treating Alzheimer's disease patients by dissociating the β-amyloid-α7 nAChR complexes that are present in human brains and that constitute senile plaques.

The use of compounds resulting from the screening method according to the invention in obtaining pharmaceutical compositions is more especially intended for the prevention and/or treatment of Alzheimer's disease patients.

“Alzheimer's disease” is understood as being a fatal neurodegenerative disease affecting memory and mental function with, in particular, deterioration of language, disturbance of complicated movements and disorders of orientation in time and space. These cognitive disorders are associated with two characteristic neuropathological lesions—senile plaques and neurofibrillary degeneration—which allow its definitive diagnosis post-mortem.

The present invention is illustrated by the following figures, without being limited thereby:

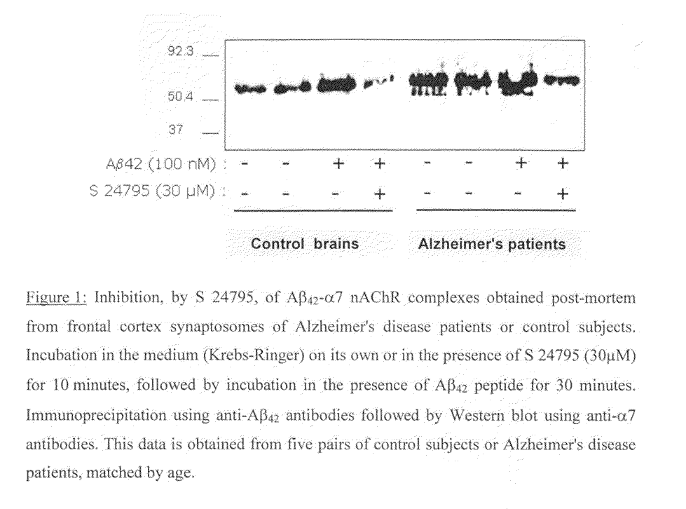

FIG. 1: Western blot illustrating the inhibition, by S 24795, of Aβ₄₂-α7 nAChR complexes obtained post-mortem from frontal cortex synaptosomes of Alzheimer's disease patients or control subjects. The Aβ₄₂-α7 nAChR complexes are incubated in the medium (Krebs-Ringer) on its own or in the presence of S 24795 (30 μM) for 10 minutes, followed by incubation in the presence of Aβ₄₂ peptide for 30 minutes.

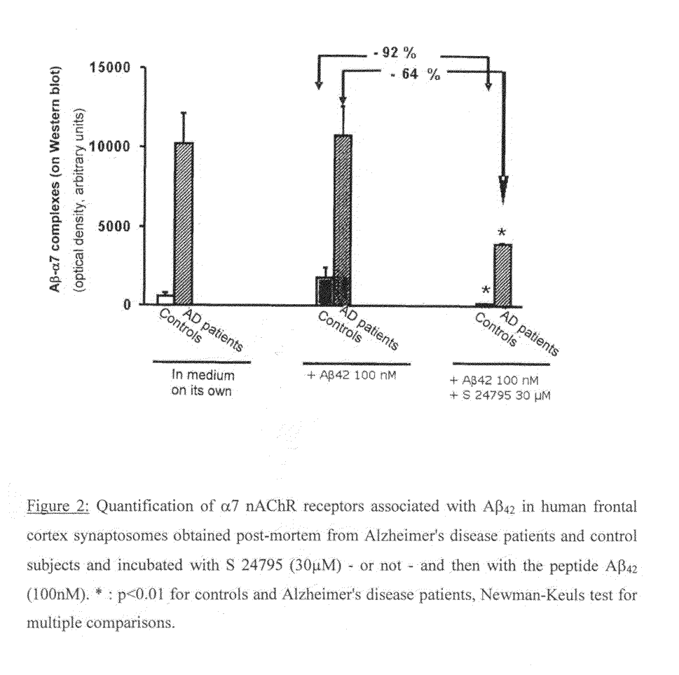

FIG. 2: Quantification of the inhibition of the formation of Aβ₄₂-α7 nAChR complexes in human frontal cortex synaptosomes obtained post-mortem from Alzheimer's disease patients and control subjects and incubated with S 24795 (30 μM)—or not—and then with Aβ₄₂ peptides (100 nM). *: p<0.01 for controls and Alzheimer's disease patients, Newman-Keuls test for multiple comparisons.

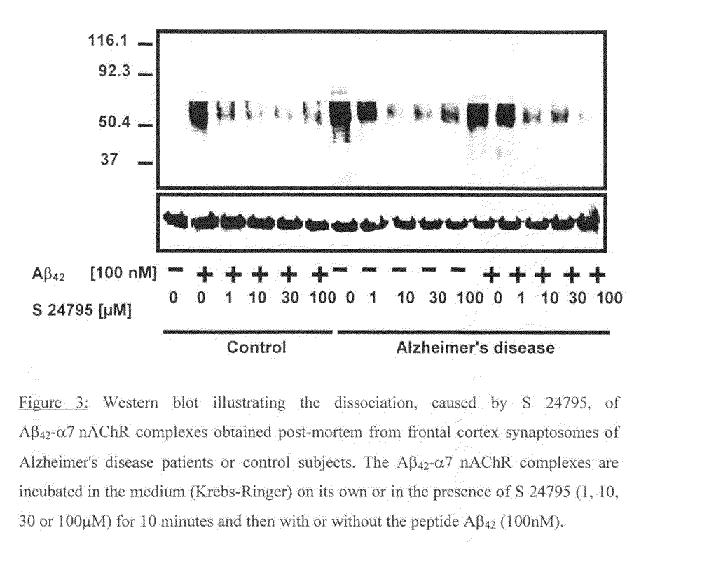

FIG. 3: Western blot illustrating the dissociation, caused by S 24795, of Aβ₄₂-α7 nAChR complexes obtained post-mortem from frontal cortex synaptosomes of Alzheimer's disease patients or control subjects. The Aβ₄₂-α7 nAChR complexes are incubated in the medium (Krebs-Ringer) on its own or in the presence of S 24795 (1, 10, 30 or 100 μM) for 10 minutes and then in the presence or absence of Aβ₄₂ (100 nM).

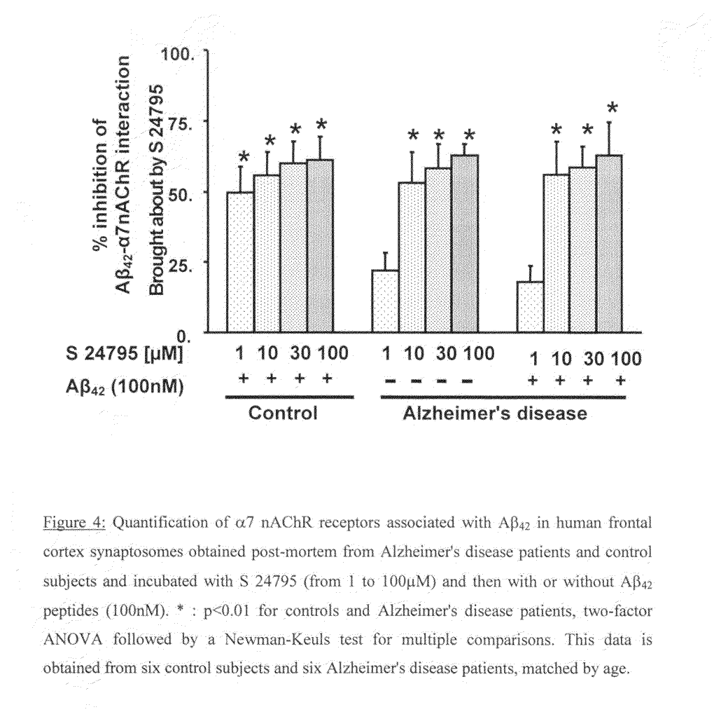

FIG. 4: Quantification of the dissociation of the interaction of α7 nAChR receptors associated with Aβ₄₂ in human frontal cortex synaptosomes obtained post-mortem from Alzheimer's disease patients and control subjects and incubated with S 24795 (from 1 to 100 μM) and then in the presence or absence of Aβ₄₂ (100 nM). *: p<0.01 for controls and Alzheimer's disease patients, Newman-Keuls test for multiple comparisons.

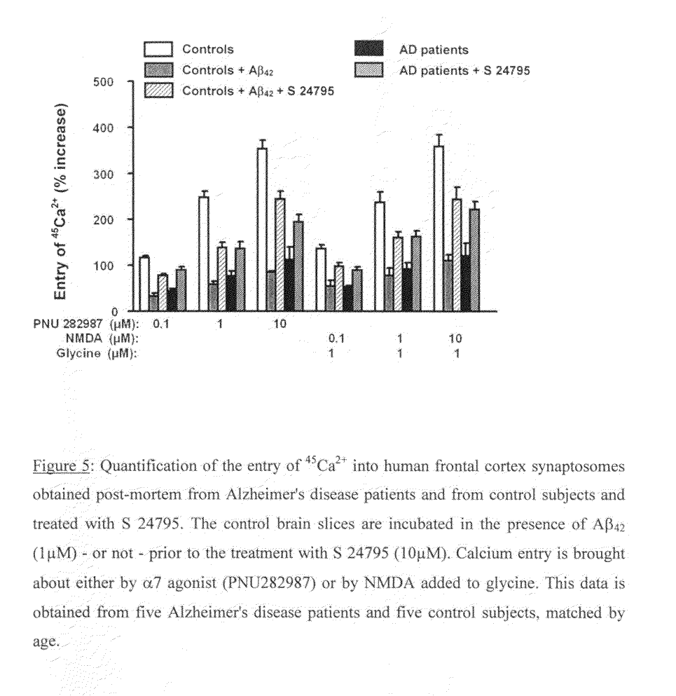

FIG. 5: Quantification of the entry of ⁴⁵Ca²⁺ into human frontal cortex synaptosomes obtained post-mortem from Alzheimer's disease patients and from control subjects and treated with S 24795. The control brain slices are incubated in the presence of Aβ₄₂ (1 μM)—or not—prior to the treatment with S 24795 (10 μM). Calcium entry is brought about either by α7 agonist (PNU282987) or by NMDA added to glycine.

### I. MATERIALS AND METHODS

### I.1) Patients

Post-mortem human frontal cortices obtained from Alzheimer's disease patients and healthy control subjects are obtained from brain banks (Harvard Brain Tissue Resource Center and Analytical Biological Services).

The patients and controls included in the study were between 50 and 90 years of age.

The controls were people who, during their life, showed no cognitive disorders or manifest signs of loss of memory.

In addition, the Alzheimer's disease patients were divided into two sub-groups having, or not having, associated vascular pathologies. Only the brains of patients without associated pathology were used in the present study.

It is to be noted that the diagnosis of Alzheimer's disease was confirmed by the immunohistochemical method of the National Institute on Aging and of the Reagan Institute Working Group on Diagnostic Criteria for the Neurological Assessment of Alzheimer's disease in patients who exhibited the clinical symptoms.

### I.2) Preparation of the Cortices

In order to avoid any post-mortem artefacts, the cortices removed in the context of the study come from people whose death occurred within the 15 hours prior to that removal. The removed cortices are stored at −80° C. until they are used in the screening method according to the invention.

### I.3) Storage of the Cortices

After removal, the cortices are cryoprotected for 2 weeks in 0.2M sodium phosphate buffer (NaH₂PO₄.2H₂O/NaH₂PO₄.12H₂O, pH 7.4) containing 20% (w/v) sucrose. They are then frozen for 1 minute in isopentane maintained at a temperature of −30° C. in solid carbon dioxide. Finally, 5-μm-thick sections, made in a cryostat thermostatically controlled at −30° C. (Super Frost Plus Fisher), are placed in a 0.02M PBS buffer and then stored at 4° C.

### I.4) Preparation of Synaptosomes

100 mg of post-mortem frontal cortex ground on ice are homogenised in 10 volumes of 10 mM HEPES pH 7.4 maintained on ice and oxygenated in the presence of 0.32 mM sucrose and 0.1 mM EDTA and then mixed in a Teflon/glass tissue grinder at 4° C. in a homogenisation solution containing 25 mM HEPES pH 7.5, 1 mM EDTA, 50 μg/ml of leupeptin, 10 μg/ml of aprotinin, 2 μg/ml of soy trypsin inhibitor, 0.04 mM PMSF, a mixture of phosphatase inhibitor proteins, and 0.2% 2-mercaptomethanol.

The homogenate is centrifuged, firstly, at 1000 g and 4° C. for 10 minutes. The supernatant obtained in that first centrifugation is centrifuged for a second time at 15000 g for 30 minutes to obtain a sediment of synaptosomes.

The sediment of synaptosomes is washed twice by suspension in 10 ml of Krebs-Ringer solution, maintained on ice, comprising 25 mM HEPES pH 7.4, 118 mM NaCl, 4.8 mM KCl, 25 mM NaHCO₃, 1.3 mM CaCl₂, 1.2 mM MgSO₄, 1.2 mM KH₂PO₄, 10 mM glucose, 100 μM ascorbic acid, 50 μg/ml of leupeptin, 10 μg/ml of aprotinin, 2 μg/ml of soy trypsin inhibitor, 0.04 mM PMSF and a mixture of phosphatase inhibitor proteins, aerated for 10 minutes using 95% O₂/5% CO₂ and then centrifuged again at 15000 g for 10 minutes at 4° C. The washed synaptosomes are then suspended in 1 ml of oxygenated Krebs-Ringer solution and the protein concentration of said synaptosome suspension is determined by the Bradford method.

### I.5) Immunoprecipitation

The human cortex synaptosomes are incubated in an oxygenated Krebs-Ringer solution in the presence of the compound S 24795 at 37° C. for 30 minutes in a total incubation volume of 500 μl. The compound S 24795 is present in the reaction mixture at a concentration of 1 μM, 10 μM, 30 μM or 100 μM. Depending on the experiments being carried out, the synaptosomes are also incubated in the presence of 100 nM Aβ₄₂ or in the presence of the vehicle. The reaction is stopped by diluting with 1.5 ml of a solution of 1 mM EDTA maintained on ice—calcium ion Ca²⁺—without Krebs-Ringer solution and then centrifuging for 10 minutes at 15000 g and 4° C. After removal of the supernatant, the sediment of synaptosomes obtained is taken up in 250 μl of immunoprecipitation buffer (25 mM HEPES pH 7.5, 200 mM NaCl, 1 mM EDTA, 50 μg/ml of leupeptin, 10 μg/ml of aprotinin, 2 μg/ml of soy trypsin inhibitor, 0.04 mM PMSF and a mixture of protein phosphatase inhibitors) containing 0.5% digitonin, 0.2% chelated sodium and 0.5% NP-40. After diluting the synaptosomes with 750 μl of immunoprecipitation buffer maintained on ice and centrifuging at 4° C. so as to remove the insoluble residues, the Aβ₄₂-α7 nAChR complexes are isolated by immunoprecipitation using anti-Aβ₄₂ antibodies, incubated in their presence for 16 hours at 4° C. and concentrated by incubation for 2 hours in the presence of 25 μl of A/G-conjugated agarose beads (Cai et al. 1999, Wang et al. 2000, Jin et al. 2001).

### I.6) Electrophoresis and Western Blot

After three washings with 1 ml of phosphate buffer saline solution pH 7.2 followed by centrifugation, the isolated Aβ₄₂-α7 nAChR complexes are dissolved in 100 μl of SDS-PAGE buffer (62.5 mM Tris-HCl pH 6.8, 10% glycerol, 2% SDS, 5% 2-mercapto-ethanol, 0.1% bromophenol blue) in the hot state for 5 minutes. The complexes are then placed on a 8-16% SDS-polyacrylamide electrophoresis gel.

Anti-α7 nAChR monoclonal antibodies are used in the Western Blot analysis and then revealed by chemoluminescence. The intensity of the bands obtained is analysed by densitometry in order to quantify the effects of the compounds, as a function of their dose, on the amount of Aβ₄₂-α7 nAChR complexes that are present.

### I.7) Anti-Amyloid Compound

The compound S 24795 is used as anti-amyloid agent in the ex vivo screening method protocol according to the invention.

The compound S 24795 is a pyridine compound used as a mnemocognitive facilitator capable of improving cognitive processes and/or of opposing the cognitive disorders associated with ageing. In contrast to the mnemocognitive facilitators that act directly on the central cholinergic systems, the compound S 24795 is lacking in hypothermic activity, which activity can be troublesome in the treatment of patients suffering from neurodegenerative disease.

### I.8) Functional Recovery Method

The functional recovery experiments are carried out using brains according to I.2 obtained from patients according to I.1.

The functional recovery brought about by the treatment with S 24795 (10 μM) is evaluated with reference to the calcium influx via α7 and NMDA receptors. It was tested after 1 hour of treatment with S 24795 on control brains exposed to Aβ₄₂ (30 minutes) and on brains of Alzheimer's disease patients. The treatments using Aβ₄₂ (1 μM) and S 24795 (10 μM) are carried out on slices of cortex obtained from those brains. Synaptosomes are then prepared as described in I.4. In order to evaluate the Ca²⁺ influxes via the α7 and NMDA receptors, the synaptosomes are incubated in the presence of ⁴⁵Ca²⁺ (5 μM) for 5 minutes at 37° C. in Krebs-Ringer medium. The Ca²⁺ fluxes are brought about for α7 selective α7nAChR receptor agonist, PNU282987 (0.1, 1 and 10 μM), and for the NMDAR receptors by the addition of NMDA (0.1, 1, 10 μM) and glycine (1 μM). The reaction is stopped by the addition of Krebs-Ringer (4° C.) containing EGTA but not containing calcium. After two washings, the synaptosomes are lysed by sonication in ethanol (95%) and the radioactivity is counted by liquid scintillation spectrometry. The specificity of the calcium influxes is checked by means of the addition of selective inhibitors of the α7 receptor (α-bungarotoxin) and of the NMDA receptor (AP-5).

### II. RESULTS

The results demonstrate two types of effect of the compound S 24795.

Firstly, the screening method makes it possible to identify, when Aβ peptides are added to the brain extracts, a preventive property of S 24795 (added before the Aβ peptides) with respect to the formation of Aβ₄₂-α7 nAChR complexes. Indeed, as can be seen from FIG. 1, the addition of Aβ peptides to non-diseased human cortex synaptosomes brings about a marked increase in the amount of Aβ₄₂-α7 nAChR complexes.

When S 24795 is added to the synaptosomes before the Aβ peptides, the amount of Aβ₄₂-α7 nAChR complexes formed is reduced by 92%, which shows that S 24795 (at 30 μM) prevents the formation of these complexes caused by the Aβ peptides.

In the synaptosomes obtained from the cortices of Alzheimer's disease patients, the amount of complexes present is twenty times higher than in the non-diseased controls. The addition of Aβ peptides does not cause an increase in the amount of complexes present in the diseased tissues, the totality of the nicotinic receptors being already saturated by endogenous Aβ peptides. In this case, the screening method makes it possible to identify a curative property of S 24795 because it dissociates complexes that were formed before the death of the patient. S 24795 brings about a substantial reduction in the amount of Aβ₄₂-α7 nAChR complexes present in the diseased brains.

The results illustrated in FIG. 3 confirm the ability of the screening method to identify a compound having a curative property because, without Aβ peptides being added, this compound can bring about dissociation of the complexes already present in the diseased human tissues. Indeed, in the absence of exogenous Aβ peptides the compound S 24795 causes, even at a concentration of 1 μM, a reduction of about 22% in the Aβ₄₂-α7 nAChR complexes previously present in the diseased brain extracts. This reduction becomes substantial at concentrations of 10, 30 and 100 μM S 24795, with non-dissociated complexes being reduced in dose-dependent manner in the order of 63% at the highest concentration (p<0.01 2-factor ANOVA followed by a Newman-Keuls test for multiple comparisons).

This screening method accordingly makes it possible to select, in specific manner, compounds that on the one hand are capable of preventing the formation of complexes, which is applicable to early disease stages, where the action of the compounds is preventive, and on the other hand are capable of dissociating the complexes already present, which is applicable to advanced or indeed severe disease stages, where the action of the compounds is curative.

Dissociation of the Aβ₄₂-α7 nAChR complexes will prevent the excessive intraneuronal accumulation of Aβ₄₂-α7 nAChR complexes and consequently oppose the neuronal death due to those deposits.

Carrying out the screening method according to the invention using human brain synaptosomes avoids the artefacts and false positives associated with the differences between animal species and humans. In addition, the ex vivo procedure of this screening method makes it possible to achieve rapidity and repeatability in identifying compounds that are capable of dissociating complexes of β-amyloid peptides with nicotinic acetylcholine receptors. Finally, as this screening method employs biological material representing a severe, fixed stage of Alzheimer's disease, it accordingly makes it possible to select and identify compounds that will act at a final stage of the disease. The screening method according to the invention makes it possible to identify compounds that can be used in the curative treatment of neurodegenerative diseases, especially Alzheimer's disease.

Furthermore, a specific experiment was conducted in order to evaluate possible functional recovery after dissociation of the Aβ₄₂-α7 nAChR complexes. This experiment shows that the dissociation of the Aβ₄₂-α7 nAChR complexes brought about by S 24795 allows recovery of certain functionalities of the α7 nAChR receptors and glutamate receptors of the NMDAR type. In comparison with the synaptosomes of control subjects, the entry of calcium via α7 nAChR and NMDAR into the synaptosomes of Alzheimer's disease patients is, in fact, greatly reduced (35% of the control values) (FIG. 5). This reduction in the entry of calcium is clearly due to the formation of the Aβ₄₂-α7 nAChR complexes because the addition of Aβ to brain slices of control subjects brings about a reduction in the entry of calcium to a level comparable to that of the patients. Treatment with S 24795, by opposing the action of Aβ in the control brains, shows substantial re-establishment of that Ca²⁺ entry.

More remarkably, treatment, with S 24795, of the complexes already formed in Alzheimer's disease patients brings about a substantial increase in Ca²⁺ entry of the order of 75% compared to the brains of untreated Alzheimer's disease patients. FIG. 5 shows that, after treatment, with S 24795, of the brains of Alzheimer's disease patients and of controls pretreated with Aβ, Ca²⁺ entry reaches comparable levels corresponding to about 65% of the control values without Aβ.

It is accordingly demonstrated by this experiment that the dissociation, brought about by S 24795, of Aβ₄₂-α7 nAChR complexes in diseased tissue obtained from Alzheimer's disease patients makes possible post-mortem restoration of certain cellular functionalities—in this particular case calcium entry.

This experiment accordingly underlines the therapeutic value of this screening method.

1-16. (canceled)

17. A method for screening compounds which dissociate or prevent complexes of β-amyloid peptides and nicotinic acetylcholine receptors derived from human brains comprising the following steps:
incubating complexes of β-amyloid peptides and nicotinic acetylcholine receptors in the presence or absence of a compound under test;
determining the amount of non-dissociated complexes; and
evaluating the difference in the amount of non-dissociated complexes in the absence of a compound relative to the non-dissociated complexes in the presence of a compound under test.

18. The method of claim 17, which identifies compounds which have curative or preventive properties in β-amyloid associated neurodegenerative diseases.

19. The method of claim 17, wherein the nicotinic acetylcholine receptors are of the α7 type.

20. The method of claim 17, wherein the β-amyloid peptides are selected from Aβ₃₉, Aβ₄₀, Aβ₄₁, Aβ₄₂ and Aβ₄₃.

21. The method of claim 17, wherein the complexes of β-amyloid peptides and nicotinic acetylcholine receptors are derived from human cortices or hippocampi.

22. The method of claim 17, wherein the dissociation of complexes of β-amyloid peptides and nicotinic acetylcholine receptors is determined by immunohistochemistry.

23. The method of claim 22, wherein the non-dissociated complexes are isolated using anti-β-amyloid peptide antibodies.

24. The method of claim 23, wherein the antibodies are directed to β-amyloid peptides selected from Aβ₃₉, Aβ₄₀, Aβ₄₁, Aβ₄₂ and Aβ₄₃.

25. The method of claim 23, wherein the non-dissociated complexes are identified using antibodies directed to nicotinic acetylcholine receptors.

26. The method of claim 25, wherein the non-dissociated complexes are identified using anti-α7 nicotinic acetylcholine receptor antibodies.

27. A compound selected from those identified by the screening method of claim 17 to be capable of dissociating or preventing complexes of β-amyloid peptides and nicotinic acetylcholine receptors derived from human brains.

28. A compound of claim 27 which is 1-(4-bromophenyl)-2-(1-methyl-2-pyridiniumyl)-1-ethanone.

29. A pharmaceutical composition comprising one or more compounds of claim 27 and one or more pharmaceutically acceptable excipients.

30. A method for the prevention and/or treatment of neurodegenerative diseases comprising administering a pharmaceutically effective amount of a compound of claim 27.

31. The method of claim 30 wherein the neurodegenerative disease is Alzheimer's disease.
